(19) United States
(12) Patent Application Publication
(10) Pub. No.: US 2015/0343075 A1
(43) Pub. Date: Dec. 3, 2015

(54) PLACEBO FORMULATIONS AND USES THEREOF

(71) Applicant: Aimmune Therapeutics, Brisbane, CA (US)

(72) Inventor: Howard V. Raff, Mill Valley, CA (US)

(21) Appl. No.: 14/728,935

(22) Filed: Jun. 2, 2015

Related U.S. Application Data
(60) Provisional application No. 62/006,699, filed on Jun. 2, 2014.

(51) Int. Cl. A61K 47/46; A61K 47/12; A61K 47/02; A61K 47/36; A61K 47/38

(52) U.S. Cl. CPC A61K 47/46; A61K 47/36; A61K 47/38; A61K 47/02; A61K 47/12

(57) ABSTRACT

The present technology relates to placebo compositions and their use in clinical trials for oral immunotherapy of peanut allergies. Therapy can be treatment or desensitization of peanut allergies. The placebo formulation is used as a control compared to an active ingredient formulation. Further, the present technology relates to methods for the preparation of the placebo compositions.

### CROSS-REFERENCE TO RELATED APPLICATION

The present application claims priority to U.S. Provisional Patent Application No. 62/006,699, filed Jun. 2, 2014, entitled “PLACEBO FORMULATIONS AND USES THEREOF,” which is incorporated herein by reference in its entirety.

### TECHNICAL FIELD

The present technology relates generally to placebo formulations, methods of manufacturing placebo formulations and uses for placebo formulations. In particular, several embodiments are directed to placebo formulations for oral administration in immunotherapy of subjects affected by allergies.

### BACKGROUND

Allergies, or a body's immunological reaction to a foreign substance (e.g., insects, foods, latex, drugs, etc.), can affect both humans and animals. The severity of allergic reactions can vary between individuals and can range from between mild irritation to anaphylaxis, which can be so severe as to be life threatening.

Allergic reactions result when a subject's immune system responds to an allergen (e.g., a foreign substance). Typically, there is no allergic reaction the first time a subject is exposed to a particular allergen. However, it is the initial response to an allergen that primes the system for subsequent allergic reactions in some individuals. In particular, the allergen is taken up by antigen presenting cells (APCs; e.g., macrophages and dendritic cells) that degrade the allergen and then display allergen fragments to T-cells. T-cells, in particular CD4+ “helper” T-cells, respond by secreting a collection of cytokines that have effects on other immune system cells. The profile of cytokines secreted by responding CD4+ T-cells determines whether subsequent exposures to the allergen will induce allergic reactions. Two classes of CD4+ T-cells (Th1 and Th2; T-lymphocyte helper type) influence the type of immune response that is mounted against an allergen.

The Th1-type immune response involves the stimulation of cellular immunity to allergens and infectious agents and is characterized by the secretion of IL-2, IL-6, IL-12, IFN-gamma, and TNF-beta by CD4+ T helper cells and the production of IgG antibodies. Exposure of CD4+ T-cells to allergens can also activate the cells to develop into Th2 cells, which secrete IL-4, IL-5, IL-10, and IL-13. IL-4 production stimulates maturation of B cells that produce IgE antibodies specific for the allergen. These allergen-specific IgE antibodies attach mast cell and basophil receptors, where they initiate a rapid immune response to the next exposure to allergen. When the subject encounters the allergen a second time, the allergen is quickly bound by these surface-associated IgE molecules, resulting in the release of histamines and other substances that trigger allergic reactions. Subjects with high levels of IgE antibodies are known to be particularly prone to allergies.

### DETAILED DESCRIPTION

### I. Overview

During the past decade, much has been learned about allergens in peanut. Peanuts are commonly associated with severe reactions, including life threatening anaphylaxis. The current standard of care in management of food allergy is dietary avoidance of the food and education of the subject/family in the acute management of an allergic reaction. The burden of avoidance and constant fear of accidental exposure negatively impacts the health-related quality of life for both subjects and their families. Quality of life surveys indicate that families with children having food allergies have significant impact on food preparation, social activities, finding appropriate childcare, school attendance, and level of stress among other things.

Currently, the only treatment for peanut allergy is a peanut-free diet and ready access to self-injectable epinephrine. However, strict avoidance diets can be complicated due to difficulty in interpreting labels and by the presence of undeclared or hidden allergens in commercially prepared foods. Accidental ingestions are unfortunately common, with up to 50% of food-allergic subjects having an allergic reaction over a two-year period. Allergic reactions to peanut can be severe and life threatening, and peanut and/or tree nut allergies account for the vast majority of fatal food-induced anaphylaxis. This combination of strict avoidance diets, the high incidence of accidental exposures, and the risk of severe or even fatal reactions with accidental exposures adds a tremendous burden and stress on subjects and their families. Further complicating matters is the fact that only about 20% of children will outgrow peanut allergy, meaning that the majority of people with peanut allergy will have it for the rest of their lives. If we couple the rising prevalence and increased consumption of peanut in Western countries with the facts that only approximately 1 in 5 will outgrow their allergy, that allergic reactions have the potential to be severe or even fatal, and that accidental exposures are common, developing an effective treatment for peanut allergy becomes even more imperative.

Specific immunotherapy for food allergy, in particular peanut allergy, in the forms of oral immunotherapy (OIT) and sublingual immunotherapy (SLIT) has been studied in recent years and has demonstrated encouraging safety and efficacy results in early clinical trials, including beneficial immunologic changes. OIT has shown evidence for inducing desensitization in most subjects with immunologic changes over time indicating progression toward clinical tolerance.

Peanut OIT: In Jones et al., peanut allergic children underwent an OIT protocol consisting of an initial dose escalation day, bi-weekly build-up (to 2 g) and daily maintenance phase followed by an OFC. After tolerating less than 50 mg peanut protein during an oral food challenge (OFC) at baseline, 27 of the 29 subjects ingested 3.9 g of peanut protein at the completion of OIT protocol.

Recently, Dr. Wesley Burks. (American Academy of Allergy, Asthma, and Immunology National Conference. Orlando, Fla., Mar. 6, 2012) presented work showing that 10 children with PA completed an OIT protocol and underwent an oral food challenge (OFC) 4 weeks after cessation of oral intake of peanut to evaluate the development of clinical “sustained unresponsiveness”. Three out of 10 subjects passed the OFC; the authors considered these subjects as clinically tolerant. Over the course of treatment, peanut IgE levels lower than 85 kU/L at a time point of 3 months into OIT was predictive of subjects who became immune tolerant.

A multi-center double-blinded randomized placebo-controlled study reported by Varshney, et al., examined twenty-eight subjects. Three subjects withdrew early in the study because of allergic side effects. After completing up-dosing, a double-blind placebo-controlled food challenge was performed, in which all remaining peanut OIT subjects (n=16) ingested the maximum cumulative dose of 5000 mg (approximately 20 peanuts), whereas placebo subjects (n=9) could tolerate only a median cumulative dose of 280 mg (range, 0-1900 mg; p<0.001). In contrast with the placebo group, the peanut OIT group showed reductions in skin prick test size (P<0.001) and increases in peanut-specific IgG4 (P<0.001). Peanut OIT subjects had initial increases in peanut-specific IgE (P<0.01) but did not show significant change from baseline by the time of oral food challenge.

As disclosed herein, formulations of peanut flour comprising characterized peanut allergens that may be formulated into a pharmaceutical composition have been developed. These presently disclosed formulations, when administered to a patient according to a treatment regimen provided herein, provide oral immunotherapy (OIT) for subjects that are allergic to peanuts. Following treatment, subjects administered an oral food challenge (OFC) are partially or fully desensitized to peanuts.

A major challenge associated with double-blind clinical trials where the active compositions are colored due to the active ingredient (e.g., peanut flour), is generating a placebo that cannot be differentiated from the active ingredient-containing compositions in color and/or texture.

Provided herein are compositions (i.e., formulations) for oral immunotherapy of peanut flour protein. Also provided herein are placebo compositions (i.e., formulations) for use in clinical trials for oral immunotherapy of peanut flour protein wherein the placebo compositions are color-matched and texture-matched to compositions containing an active ingredient.

Various aspects of the present technology provide placebo formulations, methods of manufacturing placebo formulations and uses thereof. For example, in one embodiment, the present technology provides a placebo composition that is matched in color and texture compared to a composition comprising peanut flour. In some embodiments, the placebo composition can comprise one or more glidants, one or more lubricants, one or more diluents, and optionally, one or more colorants. For example, in another embodiment, a placebo composition that is matched in color and texture compared to a composition comprising peanut flour, can comprise one or more glidants, one or more lubricants, one or more diluents, and one or more colorants.

Specific details of several embodiments of the technology are described below in the Detailed Description and the Examples. Although many of the embodiments are described below with respect to compositions (i.e., formulations) for oral immunotherapy and/or for use in clinical trials for oral immunotherapy of peanut flour protein, other applications and other embodiments in addition to those described herein are within the scope of the technology. Additionally, several other embodiments of the technology can have different components or procedures than those described herein. A person of ordinary skill in the art, therefore, will accordingly understand that the technology can have other embodiments with additional components, or the technology can have other embodiments without several of the aspects shown and described below.

### II. Definitions

Unless defined otherwise, all technical and scientific terms used herein have the same meaning as is commonly understood by one of skill in the art to which the present technology described herein belong. All patents and publications referred to herein are incorporated by reference.

The term “animal”, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

The term “antigen”, as used herein, refers to a molecule that elicits production of an antibody response (i.e., a humoral response) and/or an antigen-specific reaction with T-cells (i.e., a cellular response) in an animal.

The term “allergen”, as used herein, refers to a subset of antigens which elicit the production of IgE in addition to other isotypes of antibodies. The terms “allergen”, “natural allergen”, and “wild-type allergen” may be used interchangeably. Some examples of allergens for the purpose of the present technology are protein allergens.

The phrase “allergic reaction”, as used herein, relates to an immune response that is IgE mediated with clinical symptoms primarily involving the cutaneous (e.g., uticana, angiodema, pruritus), respiratory (e.g., wheezing, coughing, laryngeal edema, rhinorrhea, watery/itching eyes), gastrointestinal (e.g., vomiting, abdominal pain, diarrhea), and cardiovascular (i.e., if a systemic reaction occurs) systems. For the purposes of the present technology, an asthmatic reaction is considered to be a form of allergic reaction.

The phrase “anaphylactic allergen”, as used herein, refers to a subset of allergens that are recognized to present a risk of anaphylactic reaction in allergic individuals when encountered in its natural state, under natural conditions. For example, for the purposes of the present technology, pollen allergens, mite allergens, allergens in animal dander or excretions (e.g., saliva, urine), and fungi allergens are not considered to be anaphylactic allergens. On the other hand, food allergens, insect allergens, and rubber allergens (e.g., from latex) are generally considered to be anaphylactic allergens. Food allergens, in particular, are anaphylactic allergens for use in the practice of the present technology. In particular, nut allergens (e.g., from peanut, walnut, almond, pecan, cashew, hazelnut, pistachio, pine nut, brazil nut, etc.), dairy allergens (e.g., from egg, milk, etc.), seed allergens (e.g., from sesame, poppy, mustard, etc.), soybean, wheat, and fish allergens (e.g., from shrimp, crab, lobster, clams, mussels, oysters, scallops, crayfish, etc.) are anaphylactic food allergens according to the present technology. Particularly interesting anaphylactic allergens are those to which reactions are commonly so severe as to create a risk of death.

The phrase “anaphylaxis” or “anaphylactic reaction”, as used herein, refers to a subset of allergic reactions characterized by mast cell degranulation secondary to cross-linking of the high-affinity IgE receptor on mast cells and basophils induced by an anaphylactic allergen with subsequent mediator release and the production of severe systemic pathological responses in target organs, e.g., airway, skin digestive tract, and cardiovascular system. As is known in the art, the severity of an anaphylactic reaction may be monitored, for example, by assaying cutaneous reactions, puffiness around the eyes and mouth, vomiting, and/or diarrhea, followed by respiratory reactions such as wheezing and labored respiration. The most severe anaphylactic reactions can result in loss of consciousness and/or death.

The phrase “antigen presenting cell” or “APC”, as used herein, refers to cells which process and present antigens to T-cells to elicit an antigen-specific response, e.g., macrophages and dendritic cells.

When two entities are “associated with” one another as described herein, they are linked by a direct or indirect covalent or non-covalent interaction. Preferably, the association is covalent. Desirable non-covalent interactions include, for example, hydrogen bonding, van der Walls interactions, hydrophobic interactions, magnetic interactions, etc.

The phrase “decreased anaphylactic reaction”, as used herein, relates to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting).

The term “epitope”, as used herein, refers to a binding site including an amino acid motif of between approximately six and fifteen amino acids which can be bound by an immunoglobulin (e.g., IgE, IgG, etc.) or recognized by a T-cell receptor when presented by an APC in conjunction with the major histocompatibility complex (MHC). A linear epitope is one where the amino acids are recognized in the context of a simple linear sequence. A conformational epitope is one where the amino acids are recognized in the context of a particular three dimensional structure.

An allergen “fragment” according to the present technology is any part or portion of the allergen that is smaller than the intact natural allergen. In certain embodiments of the present technology, the allergen is a protein and the fragment is a peptide.

The phrase “immunodominant epitope”, as used herein, refers to an epitope which is bound by antibody in a large percentage of the sensitized population or where the titer of the antibody is high, relative to the percentage or titer of antibody reaction to other epitopes present in the same antigen. In one embodiment, an immunodominant epitope is bound by antibody in more than 50% of the sensitive population and, in further examples, more than 60%, 70%, 80%, 90%, 95%, or 99%.

The phrase “immunostimulatory sequences” or “ISS”, as used herein, relates to oligodeoxynucleotides of bacterial, viral, or invertebrate origin that are taken-up by APCs and activate them to express certain membrane receptors (e.g., B7-1 and B7-2) and secrete various cytokines (e.g., IL-1, IL-6, IL-12, TNF). These oligodeoxynucleotides contain unmethylated CpG motifs and when injected into animals in conjunction with an antigen, appear to skew the immune response towards a Th1-type response. See, for example, Yamamoto et al., Microbiol. Immunol. 36:983, 1992; Krieg et al., Nature 374:546, 1995; Pisetsky, Immunity 5:303, 1996; and Zimmerman et al., J. Immunol. 160:3627, 1998.

“Absorption” typically refers to the process of movement of a delivered substance from the gastrointestinal tract into a blood vessel.

As used herein, the terms “comprising.” “including,” and “such as” are used in their open, non-limiting sense.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. In another embodiment, the use of the term “about” indicates that values slightly outside the cited values, i.e., plus or minus 0.1% to 5%, which are also effective and safe. In another embodiment, the use of the term “about” indicates that values slightly outside the cited values, i.e., plus or minus 0.1% to 2%, which are also effective and safe.

“Isolated” (used interchangeably with “substantially pure”) when applied to polypeptides means a polypeptide or a portion thereof, which has been separated from other proteins with which it naturally occurs. Typically, the polypeptide is also substantially (i.e., from at least about 70% to about 99%) separated from substances such as antibodies or gel matrices (polyacrylamide) which are used to purify it.

### III. Compositions/Formulations

Provided herein are compositions (i.e., formulations) for oral immunotherapy of peanut flour protein.

Also provided herein are placebo compositions (i.e., formulations) for use in clinical trials for oral immunotherapy of peanut flour protein wherein the placebo compositions are color-matched and texture-matched to compositions containing an active ingredient.

Various aspects of the present technology provide placebo formulations, methods of manufacturing placebo formulations and uses thereof. For example, in one embodiment, the present technology provides a placebo composition that is matched in color and texture compared to a composition comprising peanut flour. In some embodiments, the placebo composition can comprise one or more glidants, one or more lubricants, one or more diluents, and optionally, one or more colorants. For example, in another embodiment, a placebo composition that is matched in color and texture compared to a composition comprising peanut flour, can comprise one or more glidants, one or more lubricants, one or more diluents, and one or more colorants.

One aspect of the present technology provides for a placebo composition that is matched in color and texture compared to a composition comprising peanut flour, and which comprises: one or more glidants in an amount of from about 0.01% to about 10% w/w of the placebo composition, one or more lubricants in an amount of from about 0.01% to about 10% w/w of the placebo composition, one or more diluents in an amount of from about 1% to about 99% w/w of the placebo composition, and one or more caramel colorants in an amount of from about 0.01% to about 10% w/w of the placebo composition.

Also provided herein is a placebo composition that is matched in color and texture compared to a composition comprising peanut flour, comprising: one or more glidants in an amount of from about 0.4% to about 1.5% w/w of the placebo composition, one or more lubricants in an amount of from about 0.4% to about 1.5% w/w of the placebo composition, one or more diluents in an amount of from about 5% to about 20% w/w of the placebo composition, and one or more caramel colorants in an amount of from about 0.20% to about 3% w/w of the placebo composition.

Further provided herein is a placebo composition that is matched in color and texture compared to a composition comprising peanut flour, comprising one or more glidants, one or more lubricants, one or more diluents, and optionally, one or more colorants, and a Hydroxypropyl Methyl Cellulose (HPMC) capsule shell. For example, provided herein is a placebo composition that is matched in color and texture compared to a composition comprising peanut flour, comprising one or more diluents, one or more colorants, and a HPMC capsule shell.

In yet another embodiment, provided herein is a placebo composition that is matched in color and texture compared to a composition comprising peanut flour, comprising: one or more glidants in an amount of from about 0.01% to about 10% w/w of the placebo composition, one or more lubricants in an amount of from about 0.01% to about 10% w/w of the placebo composition, one or more diluents in an amount of from about 1% to about 99% w/w of the placebo composition, one or more caramel colorants in an amount of from about 0.01% to about 10% w/w of the placebo composition, and a HPMC capsule shell.

In yet a further embodiment, provided herein is a placebo composition that is matched in color and texture compared to a composition comprising peanut flour, comprising: one or more diluents in an amount of from about 1% to about 99% w/w of the placebo composition, one or more caramel colorants in an amount of from about 0.01% to about 10% w/w of the placebo composition, and a HPMC capsule shell.

In another embodiment, provided herein is a placebo composition that is matched in color and texture compared to a composition comprising peanut flour, comprising one or more glidants in an amount of from about 0.4% to about 1.5% w/w of the placebo composition, one or more lubricants in an amount of from about 0.4% to about 1.5% w/w of the placebo composition, one or more diluents in an amount of from about 5% to about 20% w/w of the placebo composition, one or more caramel colorants in an amount of from about 0.20% to about 3% w/w of the placebo composition, and a HPMC capsule shell.

Various aspects of the present technology further provide for the use of placebo compositions, such as the placebo compositions described herein, in clinical trials as a placebo composition for treatment of peanut allergies. In some embodiments, placebo compositions described herein may be used in a clinical trial for desensitization of peanut allergies.

The placebo compositions in accordance with aspects of the present technology may be formulated for oral administration. For example, the composition can be formulated as a capsule, a tablet, a mini-tablet, a powder, or a sprinkle. In some embodiments, the composition may be a capsule comprising a HPMC capsule shell (e.g., Capsugel).

In some embodiments, the one or more diluents to be used in the compositions may be selected from the group consisting of alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), microcrystalline cellulose (e.g., Avicel®, Avicel® PH102, Avicel® PH105); silicified microcrystalline cellulse; microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), lactose (e.g., lactose monohydrate, lactose anhydrous, etc.); dicalcium phosphate; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, a starch, e.g., a natural starch such as corn starch or potato starch, a pregelatinized starch such as Colorcon (Starch 1500), National 1551 or Amijel®, or sodium starch glycolate such as Promogel® or Explotab®; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and combinations thereof. In one embodiment, the diluent is microcrystalline cellulose or starch 1500. In another embodiment, the diluents are microcrystalline cellulose and starch 1500. In yet another embodiment, the composition comprises two types of microcrystalline cellulose. For example, a placebo composition may comprise a mixture of Avicel® PH 102 and Avicel® PH 105 in a ratio of about 1:1, about 2:1, about 1:2, about 3:1, about 1:3, or any other appropriate ratio.

In one embodiment, the one or more glidants is selected from the group consisting of colloidal silicon dioxide (Cab-O-Sil), talc (e.g., Ultra Talc 4000), and combinations thereof. In one non-limiting example, the glidant is Cab-O-Sil.

Glidants may comprise from about 0.01% to about 10% w/w of the composition. For example, the glidant comprises about 0.01%, about 0.05%, about 0.1%, about 0.25%, about 0.5%, about 0.75%, about 1.0%, about 1.25%, or about 1.5% w/w of the composition. In one non-limiting embodiment, the glidant comprises about 0.5% w/w of the composition.

In certain embodiments, the target unit weight of the glidant may comprise from about 0.05 to about 5 mg/capsule. For example, the glidant may comprise about 0.725, about 2.625 or about 3.0 mg/capsule. In a non-limiting example, the glidant comprises about 2.625 mg/capsule.

In other embodiments, the one or more lubricants may be selected from the group consisting of stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and combinations thereof. In one embodiment, the lubricant is magnesium stearate. In another embodiment, the lubricant is sodium stearyl fumerate.

The composition may, for example, comprise one or more diluents in an amount of from about 1% to about 99% w/w, from about 60% to about 90%, or from about 5% to about 20% w/w of the composition. For example, the concentration of diluent may comprise about 10%, 65%, about 85%, about 87% or about 88% w/w of the composition.

The target unit weight of the diluent may, for example, comprise from about 10 to about 60 mg/capsule or from about 100 to about 700 mg/capsule. For example, the target unit weight of the diluent comprises about 14, about 52, about 125.55, about 126.99, about 394, about 446.61, about 525, or about 600 mg/capsule.

In one non-limiting example, the placebo composition comprises diluents microcrystalline cellulose and starch 1500. In a particular example, the target unit weight of starch is about 52.5 mg/capsule and the target weight of microcrystalline cellulose is about 467.25 mg.

In some embodiments, the lubricant may comprise from about 0.01% to about 10% w/w of the placebo composition. For example, the lubricant may comprise about 0.01%, about 0.05%, about 0.1%, about 0.25%, about 0.5%, about 0.75%, about 1.0%, about 1.25%, or about 1.5% w/w of the composition. In one non-limiting embodiment, the lubricant comprises about 0.5% w/w of the composition.

The target unit weight of the lubricant may, for example, comprise from about 0.05 to about 5 mg/capsule. For example, the lubricant may comprise about 0.725, about 2.625 or about 3.0 mg/capsule. In another non-limiting example, the target unit weight of the lubricant comprises about 2.625 mg/capsule.

The placebo compositions described herein may, in some embodiments, further comprise a food product.

In any of the placebo compositions described herein, the one or more colorants may be a caramel color. In one non-limiting embodiment, the caramel color comprises Sensient (05439) or Sethness (0C234). In another non-limiting embodiment, the caramel color comprises ammonia caramel, baker's caramel, confectioner's caramel, or beer caramel. In certain embodiments, the concentration of colorant in a composition described herein may be from about 0.01% to about 10% w/w of the composition.

In one embodiment, the lubricant is magnesium stearate and the concentration of lubricant in a placebo composition may be about 0.01%, about 0.05%, about 0.1%, about 0.13%, about 0.20%, about 0.25%, about 0.5%, about 0.06%, about 0.75%, about 1.0%, about 1.25%, about 1.5%, about 2.0%, about 2.5%, about 2.75%, about 2.07% about 3.0%, about 3.5% or about 4% w/w of the composition.

The target unit weight of the lubricant may, in certain embodiments, be from about 0.32 mg, about 0.7 mg, about 1.3 mg, about 7.5 mg, or about 17.8 mg color in a 450, 475, 500, 525, 550, 575, 600, 625, or 650 mg fill weight.

In one aspect, the one or more diluents may comprise a particle size of from about 10 to about 150 μm. For example, the one or more diluents may comprise a particle size of from about 20 μm to about 100 μm. In another non-limiting example, the one or more diluents comprise a mixture of particle sizes, wherein the particle sizes are from about 10 to about 150 μm. In another example, the composition comprises a 50:50 mixture of particle sizes, wherein one particle size is about 20 μm and a second particle size is about 100 μm. The placebo composition may, in some instances, further comprise an additional diluent comprising a particle size of about 100 μm. In one non-limiting embodiment, the one or more diluents comprise microcrystalline cellulose. For example, the microcrystalline cellulose comprises Avicel®, Avicel® PH 102 or Avicel® PH 105.

The placebo compositions may, in some embodiments, further comprise water.

The placebo compositions may have a loss of drying of less than about 6%, about 5.5%, about 5%, about 4.5%, about 4%, about 3.5%, about 3%, about 2.5%, or about 2% over time (e.g., 1, 2, 3, 4, 5, 6 or more months) in storage.

In a further aspect, provided herein is a kit comprising a placebo composition as described herein, one or more means to contain the composition and instructions for use. A kit may further comprise a means for administering the composition to an individual.

In one embodiment, a placebo formulation blend comprises:

| | | Target Unit Weight |
|---|---|---|
| Component: | Concentration % w/w | (mg/capsule) |
| Caramel Color | about 1.25 | about 6.6 |
| Microcrystalline | about 49.4 | about 259.2 |
| cellulose, NF (Avicel ® | | |
| PH102) | | |
| Microcrystalline | about 49.4 | about 259.2 |
| cellulose, NF (Avicel ® | | |
| PH105) | | |
| Total | 100 | 525 |

It will be understood that quantitative formulas will be adjusted depending on manufacturing fill weights.

In another embodiment, a capsulated placebo formulation that is matched in color and texture compared to a capsulated composition comprising peanut flour (100 mg) comprises:

| Material | Concentration % w/w | mg/capsule |
|---|---|---|
| Placebo blend for 100 mg | about 81.1 | about 525 |
| peanut flour capsules | | |
| Size 00, Capsule Shells | about 18.9 | about 122 |
| Total | 100 | about 647 |

In another embodiment, a placebo formulation capsule that is matched in color and texture compared to a capsulated composition comprising peanut flour (10 mg) comprises:

| Material | Concentration % w/w | Mg/capsule |
|---|---|---|
| Placebo blend for 100 mg | about 7.2 | about 40 |
| peanut flour capsules | | |
| Microcrystalline cellulose, | about 68.5 | about 360 |
| NF (Avicel ® PH102) | | |
| Size 00, Capsule Shells | about 23.9 | about 129 |
| Total | 100 | about 529 |

“Carrier materials” include any commonly used excipients in pharmaceutics and should be selected on the basis of compatibility with peanut allergen(s) and the release profile properties of the desired dosage form. Examples of carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Plasticizers” are compounds which may be used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide and combinations thereof.

“Colorants” include compounds that are used to add color to a composition. In certain embodiments, one or more colorants are added to a placebo composition so that the placebo composition matches the color of a composition comprising peanut flour proteins. In such instances, the color matching allows for the placebo compound to be indistinguishable from a composition comprising peanut flour proteins in a double-blind study. Colorants include, but are not limited to caramel color. Caramel color that may be utilized in the present formulations include, but are not limited to, Sethness powdered caramel color. In one embodiment, the powder is organic and may be manufactured from certified organic evaporated cane juice.

One non-limiting example of a caramel colorant that may be used in the present formulations is Sethness OC234 organic powdered caramel color that complies with Title 21 CFR 73.85 for Caramel. Sethness OC234 is a dark brown powder resulting from the spray drying of liquid Certified Organic Caramel Color and has a characteristic bitter, burnt sugar taste. The chemical/physical properties are as follows: Tinctorial Power, K_{0.56} (0.195-0.245); pH 1% solution (5.5-7.0); moisture, % max (4.0); particle size, % through #100 US Standard Sieve, min. (90); and a typical color intensity (0.100-0.130). Composition data is as follows for 100 g wt/wt analysis: Moisture (3.2 g); protein (0 g); total fat (0 g); cholesterol (0 mg); total carbohydrates (95.3 g); total vitamins (0 g); total nitrogen (<0.2 g); total sulfur (<0.1 g); and Ash (1.5 g).

Another non-limiting example of a caramel colorant that may be used in the present formulations is Sethness RT175 powdered caramel color that is an amorphous, dark brown material prepared from carefully controlled heat treatment of food-grade carbohydrates. It has a characteristic bitter, burnt sugar taste. The chemical/physical properties are as follows: Tinctorial Power, K_{0.36} (0.160-0.190); pH 1% solution (5.0-6.0); moisture, % max (4.0); particle size, % through #100 US Standard Sieve, min. (90); and a typical color intensity (0.078-0.096). Composition data is as follows for 100 g wt/wt analysis: Moisture (2.5 g); protein (0 g); total fat (0 g); cholesterol (0 mg); total carbohydrates (91.4 g); total vitamins (0 g); total nitrogen (0.0 g); total sulfur (0.1 g); and Ash (6.0 g).

The compositions described herein can be formulated for administration to a subject via any conventional means including, but not limited to, oral administration routes. As used herein, the term “subject” is used to mean an animal, preferably a mammal, including a human or non-human. The terms subject and subject may be used interchangeably. The formulations are for prevention and treatment of symptoms associated with exposure to limited amounts of peanut allergen in children and adults. In one embodiment, a subject is from about 4 to about 26 years of age. The formulations are also placebo formulations that are color and taste-matched to an active ingredient formulation that may be used in a double-blind clinical trial.

In some embodiments, solid dosage forms may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, pellets, or granules. In other embodiments, the formulation is in the form of a powder. In still other embodiments, the formulation is in the form of a tablet, including but not limited to, a fast-melt tablet. Additionally, formulations may be administered as a single capsule or in multiple capsule dosage form. In some embodiments, the formulation is administered in two, or three, or four, capsules or tablets or capsules.

In some embodiments, solid dosage forms, e.g., tablets, effervescent tablets, and capsules, are prepared by mixing formulation ingredients with one or more pharmaceutical excipients to form a bulk blend composition. When referring to these bulk blend compositions as homogeneous, it is meant that the particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

The pharmaceutical solid dosage forms described herein can comprise the compositions described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation. In one embodiment, some or all of the particles are coated. In another embodiment, some or all of the particles are microencapsulated. In yet another embodiment, some or all of the peanut allergens are amorphous material coated and/or microencapsulated with inert excipients. In still another embodiment, the particles are not microencapsulated and are uncoated.

Compressed tablets are solid dosage forms prepared by compacting the bulk blend formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the formulation. In other embodiments, the film coating aids in subject compliance (e.g., Opadry® coatings or sugar coating). Film coatings comprising Opadry® typically range from about 1% to about 3% of the tablet weight. In other embodiments, the compressed tablets comprise one or more excipients.

A capsule may be prepared, e.g., by placing the bulk blend formulation, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulations are placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In one aspect of the present technology, dosage forms may include microencapsulated formulations. In some embodiments, one or more other compatible materials are present in the microencapsulation material. Examples of materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with peanut allergens which sufficiently isolate peanut allergens from other non-compatible excipients. Materials compatible with peanut allergens are those that delay the release of the peanut allergens in vivo.

Examples of microencapsulation materials useful for delaying the release of the formulations include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR. Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, EudragitW S100, EudragitW RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Microencapsulated peanut allergens may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The formulations described herein are administered and dosed in accordance with good medical practice, taking into account the clinical condition of the individual subject, the site and method of administration, scheduling of administration, and other factors known to medical practitioners.

### IV. Kits

In one aspect, provided herein is a kit, comprising a placebo composition described herein, one or more means to contain the composition and instructions for use. A kit may further comprise a means for administering the composition to an individual.

The kits can thus comprise, in suitable container means, a composition as described herein. The container means of the kits will generally include at least one vial, test tube, flask, bottle, syringe and/or other container means, into which the at least one composition can be placed, and/or suitably aliquoted. The kits can include a means for containing reagent containers in close confinement for commercial sale. Such containers may include injection and/or blow-molded plastic containers into which the desired vials are retained. Kits can also include printed material for use of the materials in the kit.

Packages and kits can additionally include a buffering agent, a preservative and/or a stabilizing agent in a pharmaceutical formulation. Each component of the kit can be enclosed within an individual container and all of the various containers can be within a single package. In some embodiments, kits can be designed for cold storage or room temperature storage. Packages and kits can additionally include desiccators.

Packages and kits can further include a label specifying, for example, a product description, mode of administration and/or indication of treatment. In one aspect, the package can further include a label for use in a clinical trial for treating peanut allergies. In one aspect, the package can further include a label for use in a clinical trial for desensitizing peanut allergies.

The term “packaging material” refers to a physical structure housing the components of the kit. The packaging material can maintain the components sterilely and can be made of material commonly used for such purposes (e.g., paper, corrugated fiber, glass, plastic, foil, ampules, etc.).

The label or packaging insert can include appropriate written instructions. Kits, therefore, can additionally include labels or instructions for using the kit components. A kit can include a compound in a pack, or dispenser together with instructions for administering the compound to an individual.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, flash drive, electrical storage media such as RAM and ROM, IC tip and hybrids of these such as magnetic/optical storage media, or any other suitable medium.

### V. Methods of Use

Compositions described herein may be used in clinical trials designed to treat using oral immunotherapy (OIT) a subject suffering from a peanut allergy. The placebo compositions are formulated to match the active ingredient compositions comprising peanut flour such that they are indistinguishable to practitioners administering medication in a double-blind study.

Peanuts and peanut flour are common foods and additives found in many food products. The intended clinical use for Characterized Peanut Allergen (CPA) is found in relatively small quantities (0.5 to 4000 mg/dose) compared to quantities contained in food and will be delivered via the same route as orally ingested peanut-containing products.

Currently, preclinical studies exploring treatment modalities in food allergy animal models are limited. The principle model for induction of peanut allergy in mice is to expose mice by oral gavage to peanut proteins in the form of peanut butter, ground roasted peanuts, or purified peanut proteins, in combination with cholera toxin. After 3 to 6 weekly exposures the mice are challenged to demonstrate an allergic response. Mice may be challenged by intraperitoneal injection with sub-lethal doses of with a formulation described herein and scored for reaction severity. The intent is to demonstrate that the principle elicitors of anaphylaxis are specific Ara h proteins, rather than a combination of all peanut proteins. In an immunotherapy protocol, mice are treated with whole peanut extract, extract depleted of Ara h proteins, or with purified Ara h proteins alone. Upon challenge post treatment, changes in body temperature, symptom score and mouse mast cell protease-1 release mice may be assessed. Mice that are desensitized to further challenge may be treated with an entire extract or the Ara h protein combination.

The cellular requirements underlying peanut induced anaphylaxis may be determined explored in wild-type C57BL/6, B-cell deficient, CD40L-deficient, mast cell deficient or FcεRI ε-chain-deficient mice sensitized to peanut proteins. After intraperitoneal challenge with a formulation described herein, anaphylaxis is assessed by measurement of antigen-specific immunoglobulins (Igs), overall symptom score, body temperature, vascular permeability, mast cell mediator release and anaphylactic reactions. The B-cell, mast cell and CD40L deficient mice may be sensitized to peanut proteins as shown by production of IgE, and Th2-associated cytokines. The FcεRI ε-deficient mice may experience anaphylaxis albeit somewhat less severe than the wild-type animals.

In a model of esophago-gastro-enteropathy induced by long term feeding of peanuts to sensitized mice described by Mondoulet et al., 2012, epicutaneous immunotherapy with a formulation described herein may lessen the severity of gastro-intestinal lesions. (Mondoulet et al., 2012).

Placebo formulations are administered to a designated number of individuals in a clinical trial, but may not be readily visually distinguishable from the active ingredient compositions.

Data obtained from these models, which may demonstrate one or more of the hallmarks of human food allergic reactions, and are to be considered with respect to variability of human food allergy.

A subject treated with an active ingredient composition may exhibit a decreased anaphylactic reaction, relating to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting) following treatment. In one embodiment, a subject may exhibit a decreased anaphylactic reaction of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with an active ingredient composition described herein may exhibit a decreased humoral response and/or T cell response following treatment. In one embodiment, a subject may exhibit a decreased humoral response and/or T cell response of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with an active ingredient composition described herein may exhibit a decreased IgE response and/or a decreased mast cell response following treatment. In one embodiment, a subject may exhibit a decreased IgE response and/or a decreased mast cell response of about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

A subject treated with an active ingredient composition described herein may be better able to withstand an oral food challenge (OFC) following treatment.

A subject treated with an active ingredient composition described herein may be desensitized to peanut allergy following treatment. In one embodiment, a subject may be desensitized by about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or more compared to a subject receiving a placebo or a subject not receiving treatment.

The active ingredient compositions described herein may be administered in an escalation schedule. In one embodiment, escalating doses are administered to the subject on day 1 of treatment. For example, a subject may be administered, 1, 2, 3, 4 or 5 doses of a composition described herein on day 1. In another example, a subject may be administered 5 doses of a composition described herein in 30 minute increments on day 1. Subjects return on day 2 and receive a maximum tolerated dose. Subjects with moderate symptoms observed on day 2 may return on day 3 for the next lower dose under observation in the Clinical Research Center or monitored clinic setting. Subjects able to withstand treatment on the initial day of treatment may be administered one or more further doses of a composition described herein.

In one embodiment, a subject is further administered 1, 2, 3, 4, 5, 6, 7, 8 or 9 additional escalating doses of a composition described herein. The additional escalating doses may be administered to a subject in two-week intervals.

Following the final administration, the subject may, in some instances, be subject to an oral food challenge to determine if the subject has been desensitized to peanut allergy.

Placebo formulations are administered on the same schedule as the active ingredient compositions. A non-limiting example of a method (e.g., administration schedule) is further described in Table 1.

TABLE 1
Dose Schedule
Initial Day Escalation Schedule

| | | Cumulative Peanut |
|---|---|---|
| Dose # | Peanut Protein Dose | Protein Dose* |
| 1 | 0.5 mg | 0.5 mg |
| 2 | 1.0 mg | 1.5 mg |
| 3 | 1.5 mg | 3.0 mg |
| 4 | 3.0 mg | 6.0 mg |
| 5 | 6.0 mg | 12 mg |

*If no de-escalation

Doses are administered at a frequency standard of every 30 minutes.

Subjects at the end of the first day, tolerating less than 1.5 mg single dose, in some instances, may be considered an initial day escalation desensitization failure.

Subjects tolerating only a 1.5 mg or 3 mg single dose will go home on the greatest tolerated dose to be given daily (first dose given in Clinical Research Center under observation). All escalations will occur no sooner than 2 weeks and single dose increases in the Clinical Research Center from 1.5 mg to 3 mg to 6 mg will be attempted.

All subjects will return on Day 2 and receive their maximum tolerated dose under direct observation. Subjects with moderate symptoms observed on Day 2 will return on Day 3 for the next lower dose under observation in the Clinical Research Center or monitored clinic setting. Doses on day 2, 3 and 4 may be at least 1.5 mg or the subject, in some instances, may be considered an escalation failure.

Following the initial escalation, if a subject does not have an adverse event, the dose schedule in Table 2 is followed:

TABLE 2
Dose Schedule
Escalation Dosing

| Dose # | Dose (Protein) | Interval (weeks) | % Increase |
|---|---|---|---|
| 6 | 12 mg | 2 | |
| 7 | 20 mg | 2 | 67% |
| 8 | 40 mg | 2 | 100% |
| 9 | 80 mg | 2 | 100% |
| 10 | 120 mg | 2 | 50% |
| 11 | 160 mg | 2 | 33% |
| 12 | 200 mg | 2 | 25% |
| 13 | 240 mg | 2 | 20% |
| 14 | 300 mg | 2 | 25% |

Embodiments of dosing schedules are provided herein in the Examples.

In one embodiment of such methods, immediately prior to administration, an encapsulated capsule formulation may be broken apart and the ingredients mixed into taste-masking food.

Subjects may be monitored for onset of systemic symptoms including, for example, flushing, intensive itching on the skin, and sneezing and runny nose. Sense of heat, general discomfort and agitation/anxiety may also occur.

Provided herein is a method of desensitizing a subject suffering from a peanut allergy comprising administering one or more doses of a composition of any of the preceding claims on one more days to said subject.

In one embodiment, the subject is desensitized by at least about 2% compared to a subject administered a placebo or not receiving treatment.

In another embodiment, the subject exhibits a reduced humoral response and/or a reduced T cell response.

In another embodiment, the subject exhibits reduced anaphylaxis, a reduced mast cell response, a reduced IgE response, reduced hives, or a combination thereof.

In some embodiments, a composition described herein may be administered in conjunction with a food product.

A subject may be administered a composition described herein 1, 2, 3, 4 or 5 doses on a first day of treatment.

In one embodiment, a subject is administered 5 doses.

In another embodiment, the subject is administered said doses in 30 minute intervals.

The method may, in some instances further comprise one or more additional treatments.

In some embodiments, the one or more additional treatments comprise administration of a composition in two-week intervals.

In other embodiments, the one or more additional treatments comprise 1, 2, 3, 4, 5, 6, 7, 8, 9, or more doses of a composition.

Provided herein is a method of desensitizing a subject suffering from a peanut allergy comprising administering one or more doses of a composition of any of the preceding claims, said method comprising the following steps: (a) administering to said subject escalating doses of 0.5 mg, 1.0 mg, 1.5 mg. 3.0 mg, and 0.6 mg in 30-minute intervals on day 1 of the treatment regimen; (b) optionally, administering to said patient a maximum tolerated dose on day 2 of the treatment regimen; and (c) administering to said subject single doses of 12 mg, 20 mg, 40 mg, 80 mg, 120 mg, 160 mg, 200 mg, 240 mg, and 300 mg in two-week intervals.

In one embodiment, the method further comprises administering an oral food challenge (OFC) following completion of the treatment regimen.

Combination Therapies

The formulations and methods described herein may also be used in conjunction with other well-known therapeutic reagents that are selected for their particular usefulness against the condition that is being treated. In general, the formulations described herein and, in embodiments where combinational therapy is employed, other agents do not have to be administered in the same formulation, and may, because of different physical and chemical characteristics, have to be administered by different routes. The determination of the mode of administration and the advisability of administration, where possible, in the same formulation, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the subject and the appropriate treatment protocol. The compounds may be administered concurrently (e.g., simultaneously, essentially simultaneously or within the same treatment protocol) or sequentially, depending upon the condition of the subject, and the actual choice of compounds used. The determination of the order of administration, and the number of repetitions of administration of each therapeutic agent during a treatment protocol, is well within the knowledge of the skilled physician after evaluation of the severity of peanut allergy being treated and the condition of the subject.

It is understood that the dosage regimen to treat, prevent, or ameliorate peanut allergy, can be modified in accordance with a variety of factors. These factors include the age, weight, sex, diet, and/or medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In some embodiments, the formulation is administered with at least one other anti-histamine agent, corticosteroid, beta agonist, anti-inflammatory agent, an anti-IgE antibody (e.g., omalizumab) and/or epinephrine.

### VI. Examples

The present technology may be better understood by reference to the following non-limiting examples. The following examples are presented in order to more fully illustrate certain embodiments and should in no way be construed, however, as limiting the broad scope of the present technology. While certain embodiments of the present technology have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

### Example 1

### Placebo Formulation

This example describes preparation of a placebo composition using a caramel shade of color and a 1:1 mixture of Avicel® PH 102: Avicel® PH 105.

Materials:

Water, Ricca Lot 2203183 (USP/EP Purified)

Color—Sethness Powdered Caramel, Lot OC234/2343109A

Avicel® PH102 FMC Bio Polymer Lot P212824644

Avicel® PH105 FMC Bio Polymer Lot 51309C

Vector Fluid Bed Asset #00061

Overhead Stirrer Asset #00117

Balance Asset #00096

Procedure:

A 5% solution was prepared by dissolving 25 g color in 500 mL water. 100 g Avicel® PH102 and 100 g Avicel® PH105 mixed well in plastic bag, then placed in chamber of fluid bed. The air control knob is turned off and atomization air pressure is set to 20 psi. A spray gun is primed at a target spray rate of 50 rpm. Spray parameters were then set so that spray was on for 0.1 min, off for 0.1 min with pulse at 3 sec. The color solution was sprayed for approximately 6 min intervals followed by approximately 2 min drying. Weights of materials, machine parameters and procedures are found in Table 3.

TABLE 3

| | |
|---|---|
| Tare weight beaker | 217.776 g |
| only: | |
| Weight beaker with | 586.452 g |
| solution: | |
| Weight beaker with | 585.182 |
| solution after priming | |
| pump: | |
| Start time 10:15 | Airflow at 120 LPM |
| | Nozzle air at 20.8 PSI |
| | Pump speed at 50 RPM |
| At 1 hr/11:15: | Airflow at 235 LPM |
| | Nozzle air at 20.8 PSI |
| | Pump Speed at 50 RPM |
| At 1 hr continued: | Exhaust temp 32.3° C. |
| | Spraying stopped momentarily to examine blend |
| | Some material clumped on spray nozzle and small |
| | clump of colored material in chamber; |
| | Chamber scraped down, spray gun clump scraped |
| | into EC chamber; |
| | material spray dried w/o addition of more color |
| | for approximately 15 minutes - after clumps were |
| | broken up with spatula and stirred by hand into |
| | blend |
| EE 29 Aug. 2013 | Spraying of this batch aborted - dark, small dry |
| | clumps of material found at the bottom of |
| | chamber |
| | Process will be repeated with increased drying |
| | time between sets of shorter spraying times |
| Beaker w/color weight | 505.044 g |
| at stop point | |

Repeating procedure with these changes to parameters:

| | |
|---|---|
| Spray time set point | On 0.1 min |
| | Off 0.2 min |
| Start time 13:20: | Airflow at 187 LPM |
| | Nozzle air at 20.8 PSI |
| | Pump speed at 50 RPM |

After 49+99 cycles, the material is dried for 2-5 min, then check pump gun to be sure it is free of clumped material (dry until material flows freely as before spray starts). Tare weight beaker w/ solution after priming=591.046 g.

The process was stopped at 13:30 because gun was covered in clumped material.

Weight beaker w/ color at this point=578.113 g.

Machine trouble shooting, then restarted.

Weight beaker w/ color at restart=573.189 g.

Airflow at 120 LPM

Nozzle air at 20.6 PSI

Pump speed at 50 RPM

“A” 24 g of material removed me as possible color-match weight beaker w/ solution=535.961 g; “B” material sprayed to darker color at same parameters as described previously; and final weight of beaker w/ solution=525.688 g.

Dilutions of 24 g were made using both Avicel PH 102 and Avicel PH 105. For these dilutions, “A” refers to the 24 g sample pulled prior to the final spraying.

“B” refers to final material at the end of spraying.

TABLE 4

| | | |
|---|---|---|
| Options | C: 2.5 g blend B, 2.5 Avicel ® 105 | too light |
| for 1000 mg | D: 4 g blend B, 1 g Avicel 105 | good color, too fine |
| Placebo | E: 4 g blend B, 1 g Avicel 102 | good color, better |
| | | texture |
| | F: 3 g blend B, 2 g Avicel 105 | too light |
| Options | G: 4 g blend A, 1 g Avicel 102 | too dark |
| for 0 mg | H: 1 g blend A, 4 g Avicel 102 | slightly dark |
| placebo | I: 0.5 g blend A, 4.5 g Avicel 102 | good match |

Blend A and Blend I were used for capsule fillblend recovery study. Five (5) capsules were filled, then emptied. Weights of empty capsule, pack filled capsule and recovered material were captured. The percentage (%) recovery was calculated. Blend A was filled at a target of 725 mg into green 00 capsules (Capsugel, Lot 70912401). Target 725=600 fill+125 shell

TABLE 5

| | Empty | Filled | Material | Weight | % |
|---|---|---|---|---|---|
| | Weight | Weight | Weight | Recovered | Recovered |
| | 132 mg | 691 mg | 559 mg | 551 mg | 98.6 |
| | 125 mg | 705 mg | 580 mg | 578 mg | 99.7 |
| | 126 mg | 748 mg | 622 mg | 617 mg | 99.2 |
| | 132 mg | 708 mg | 576 mg | 573 mg | 99.5 |
| | 126 mg | 699 mg | 573 mg | 570 mg | 99.5 |
| Ave: | 128.2 mg | 710.2 mg | 582 mg | 571.8 mg | 99.3 |

Blend I was filled at a target of 649 mg into blue 00 capsule (Capsugel, Lot 70912411). Target=525 fill+124 shell=649

TABLE 6

| | Empty | Filled | Material | Weight | % |
|---|---|---|---|---|---|
| | Weight | Weight | Weight | Recovered | Recovered |
| | 116 mg | 625 mg | 509 | 502 | 98.6 |
| | 123 mg | 650 mg | 527 | 524 | 99.4 |
| | 125 mg | 664 mg | 539 | 533 | 98.9 |
| | 127 mg | 601 mg | 474 | 468 | 98.7 |
| | 126 mg | 587 mg | 461 | 460 | 99.8 |
| Ave: | 123.4 mg | 625.4 mg | 502 | 517.4 | 99.1 |

Blend I was much more difficult than blend A to get to target, capsules were filled by hand and packed as full as possible)

Samples of blends A+B were prepared as placebo for 100 mg capsules.

Samples of blends H+I were prepared as 10 mg placebo.

Samples of capsules filled w/ blends A+B were also prepared, along with active capsules of these strengths.

### Example 2

### Placebo Formulation

This example describes preparation of a placebo samples using 50-50 Avicel® 102-Avicel® 105 blend that was colored in an earlier study (see Example 1).

Materials:

Blend A from L0095

Blend B from L0095

Avicel® PH 102 Lot P212824644

TABLE 7
For 10 mg Placebo:

Lot L0095-72-1
0.5 g Blend A L0095
4.5 g Avicel ® PH 102
(this is a repeat of blend I)
Lot L0095-72-2
0.25 g Blend A
4.75 g Avicel ® PH 102

Capsules of both blends (above) were prepared using 00 Blue Opaque Casugel Lot 70912411; hand filled to max/pack-fill.

Capsules of Blends “A” and “B” were prepared using 00 Green Opaque Casugel Lot 70912401, as above. These and blend samples were labeled with Lot #s: Lot L0095-69-A and Lot L0095-69-B.

Samples of Active were also pulled and sent as blend and capsule samples for comparison: 10 mg active from Batch 054-12033C and 100 mg active from Batch 054-13037D.

Color Concentration Calculations—Ref D0125, DS 32, for am but Lot L0095-72-2.

Color Concentration for lot L0095-72-2.
0.25 g colored Avicel at 1.254% colorant
(0.25)(0.01254)=0.0031 g color

$$w / w color in sample = \frac{0.0031 g color}{5 g blend} = 0.00062 \times 100 \% = 0.062 \% color$$

### Example 3

### Placebo Formulations

The following example provides examples of formulation placebo blends for 100 mg peanut flour capsules and 10 mg peanut flour capsules.

First, a formulation Placebo Blend was prepared for 100 mg Peanut Flour Capsules:

TABLE 8

| | | | Demo Batch Size | Phase 3 |
|---|---|---|---|---|
| | % | Mg/ | (eq. to 1 sub- | (8 Sub- |
| Material | W/W | capsule | lot), g | lots), kg |
| Sethness OC234 | 1.25 | 6.6 | 68.75 | 0.55 |
| Organic Powdered | | | | |
| Caramel Color | | | | |
| Microcrystalline | 49.375 | 259.2 | 2715.625 | 21.7 |
| Cellulose, NF | | | | |
| (Avicel ® PH102) | | | | |
| Microcrystalline | 49.375 | 259.2 | 2715.625 | 21.7 |
| Cellulose, NF | | | | |
| (Avicel ® PH105) | | | | |
| Purified Water USP | * | * | 1375* | 11.0* |
| Total | 100 | 525 | 5,500 | 43.95 |

*Evaporated during the process

Formulation Placebo Capsules for Peanut Flour 100 mg were prepared as described in Table 9.

TABLE 9

| | | | Demo Batch Size | Phase 3 |
|---|---|---|---|---|
| | % | Mg/ | (9142 | (60,000 |
| Material | W/W | capsule | capsules), g | capsules), kg |
| Placebo Blend | 81.1 | 525 | 4800 | 31.50 |
| for 100 mg | | | | |
| Peanut Flour Capsules | | | | |
| Size 00, | 18.9 | 122 | 1189 | 7.32 |
| Swedish Opaque | | | | |
| Capsule Shells | | | | |
| Total | 100 | 647 | 5989 | 38.82 |

Formulation Placebo Capsules for Peanut Flour 10 mg were prepared as described in Table 10 as follows:

TABLE 10

| | | | Demo Batch Size | Phase 3 |
|---|---|---|---|---|
| | % | Mg/ | (10240 | (60,000 |
| Material | W/W | capsule | capsules), g | capsules), kg |
| Placebo Blend | 7.6 | 40 | 410 | 2.40 |
| for 100 mg | | | | |
| Peanut Flour Capsules | | | | |
| Microcrystalline | 68.5 | 360 | 3686 | 21.60 |
| Cellulose | | | | |
| (Avicel ® PH102) | | | | |
| Size 00, Blue Opaque | 23.9 | 129 | 1321 | 7.74 |
| Capsule Shells | | | | |

### Example 4

### Peanut Placebo Flour Capsule Stability Study

A 3-month stability study of 10 and 100 mg peanut placebo capsules was conducted. All capsules were stored at 25° C. and 60% relative humidity; appearance, deliverable mass and loss on drying was examined at each pull point.

TABLE 11
Appearance Results

| Time | Capsule | 100 mg | 10 mg |
|---|---|---|---|
| T = 0 | Shell | Green opaque capsule | Blue opaque capsule |
| | Fill | Beige fine powder | White to off-white, |
| | | w/clumps | fine granular powder |
| T = 1 Month | Shell | Green opaque capsule | Blue opaque capsule |
| | Fill | Beige fine powder | White to off-white, |
| | | w/clumps | fine granular powder |
| T = 3 Months | Shell | Green opaque capsule | Blue opaque capsule |
| | Fill | Beige fine powder | White to off-white, |
| | | w/clumps | fine granular powder |

Loss of drying was determined for two batches at 0, 1 and 3 months.

TABLE 12
Loss on Drying (LOD)

Batch

| | |
|---|---|
| 100 mg | 10 mg |

| | |
|---|---|
| Time (M) | LOD (%) |

| | | |
|---|---|---|
| 0 | 4.7 | 4.1 |
| 1 | 3.8 | 4.3 |
| 3 | 5.2 | 5.2 |

The following tables demonstrate the deliverable mass of the 100 mg (Tables 13-15) and 10 mg (Tables 16-18) capsules at 0, 1 and 3 months, respectively.

TABLE 13

| | Deliverable Mass, 100 mg at T = 0 | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 628.6 | 499.1 | 127.9 | 99.7 |
| 2 | 637.3 | 513.7 | 123.2 | 99.9 |
| 3 | 631.8 | 509.4 | 121.2 | 99.8 |
| 4 | 646.1 | 516.2 | 128.0 | 99.6 |
| 5 | 641.5 | 518.8 | 120.5 | 99.6 |
| 6 | 648.1 | 522.6 | 124.4 | 99.8 |
| 7 | 613.7 | 485.5 | 126.9 | 99.7 |
| 8 | 645.6 | 512.4 | 131.6 | 99.7 |
| 9 | 637.6 | 515.8 | 120.4 | 99.7 |
| 10 | 624.5 | 506.3 | 118.4 | 100.0 |
| Average | 635.5 | 510.0 | 124.3 | 99.8 |
| Std Dev | 10.9 | 10.9 | 4.2 | 0.1 |
| % RSD | 1.7 | 2.1 | 3.4 | 0.1 |

TABLE 14

| | Deliverable Mass, 100 mg at T = 1 month (M) | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 629.2 | 501.8 | 125.8 | 99.7 |
| 2 | 615.4 | 493.0 | 121.5 | 99.8 |
| 3 | 628.8 | 502.0 | 125.4 | 99.7 |
| 4 | 633.2 | 510.1 | 120.6 | 99.5 |
| 5 | 635.5 | 512.6 | 120.9 | 99.6 |
| 6 | 628.4 | 505.3 | 120.5 | 99.5 |
| 7 | 633.4 | 508.7 | 123.5 | 99.8 |
| 8 | 645.8 | 524.7 | 119.2 | 99.6 |
| 9 | 659.4 | 535.6 | 123.2 | 99.9 |
| 10 | 635.8 | 509.0 | 123.8 | 99.4 |
| Average | 634.5 | 510.3 | 122.4 | 99.7 |
| Std Dev | 11.6 | 12.1 | 2.2 | 0.2 |
| % RSD | 1.8 | 2.4 | 1.8 | 0.2 |

TABLE 15

| | Deliverable Mass, 100 mg at T = 3 M | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 623.0 | 495.3 | 124.0 | 99.3 |
| 2 | 618.0 | 496.2 | 120.7 | 99.8 |
| 3 | 639.8 | 514.6 | 122.7 | 99.5 |
| 4 | 635.5 | 513.8 | 120.0 | 99.7 |
| 5 | 637.9 | 515.3 | 122.0 | 99.9 |
| 6 | 671.0 | 543.3 | 126.0 | 99.7 |
| 7 | 637.0 | 516.5 | 120.2 | 99.9 |
| 8 | 630.5 | 511.3 | 116.8 | 99.5 |
| 9 | 636.8 | 512.2 | 122.5 | 99.6 |
| 10 | 631.7 | 504.8 | 125.4 | 99.7 |
| Average | 636.1 | 512.3 | 122.0 | 99.7 |
| Std Dev | 14.1 | 13.3 | 2.8 | 0.2 |
| % RSD | 2.2 | 2.6 | 2.3 | 0.2 |

TABLE 16

| | Deliverable Mass, 10 mg at T = 0 M | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 525.5 | 402.0 | 123.4 | 100.0 |
| 2 | 528.6 | 399.5 | 128.6 | 99.9 |
| 3 | 527.6 | 400.1 | 127.1 | 99.9 |
| 4 | 533.8 | 406.5 | 126.7 | 99.9 |
| 5 | 537.8 | 416.8 | 120.8 | 100.0 |
| 6 | 548.7 | 423.8 | 123.5 | 99.7 |
| 7 | 511.7 | 387.1 | 123.1 | 99.6 |
| 8 | 522.6 | 392.7 | 129.3 | 99.8 |
| 9 | 514.1 | 389.2 | 125.3 | 100.1 |
| 10 | 515.5 | 392.5 | 122.3 | 99.8 |
| Average | 526.6 | 401.0 | 125.0 | 99.9 |
| Std Dev | 11.5 | 11.9 | 2.8 | 0.1 |
| % RSD | 2.2 | 3.0 | 2.3 | 0.1 |

TABLE 17

| | Deliverable Mass, Batch 10 mg at T = 1 M | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 526.2 | 402.5 | 121.7 | 99.5 |
| 2 | 553.5 | 427.5 | 125.5 | 99.9 |
| 3 | 521.8 | 394.9 | 125.8 | 99.7 |
| 4 | 530.6 | 402.9 | 126.1 | 99.6 |
| 5 | 518.9 | 392.0 | 125.7 | 99.7 |
| 6 | 525.3 | 400.7 | 123.9 | 99.8 |
| 7 | 540.3 | 415.5 | 123.0 | 99.6 |
| 8 | 547.3 | 419.0 | 127.3 | 99.8 |
| 9 | 530.3 | 406.6 | 122.7 | 99.8 |
| 10 | 538.1 | 407.1 | 129.7 | 99.7 |
| Average | 533.2 | 406.9 | 125.1 | 99.7 |
| Std Dev | 11.3 | 11.0 | 2.4 | 0.1 |
| % RSD | 2.1 | 2.7 | 1.9 | 0.1 |

TABLE 18

| | Deliverable Mass, 10 mg at T = 3 M | | | |
|---|---|---|---|---|
| | Weight of | Weight | Weight of | |
| | Full Capsule | Delivered | Empty Capsule | % Deliverable |
| Sample ID | (mg) | (mg) | (mg) | Mass |
| 1 | 540.0 | 412.2 | 126.0 | 99.6 |
| 2 | 540.9 | 412.2 | 129.0 | 100.1 |
| 3 | 546.5 | 421.2 | 125.9 | 100.1 |
| 4 | 548.0 | 422.9 | 125.8 | 100.2 |
| 5 | 524.8 | 401.1 | 124.0 | 100.1 |
| 6 | 545.9 | 420.4 | 125.7 | 100.0 |
| 7 | 538.1 | 414.3 | 124.5 | 100.2 |
| 8 | 526.0 | 404.6 | 120.5 | 99.8 |
| 9 | 555.4 | 428.9 | 125.5 | 99.8 |
| 10 | 546.6 | 419.6 | 126.6 | 99.9 |
| Average | 541.2 | 415.7 | 125.4 | 100.0 |
| Std Dev | 9.7 | 8.5 | 2.2 | 0.2 |
| % RSD | 1.8 | 2.1 | 1.7 | 0.2 |

The deliverable mass of the capsules remained at over 99% over the 3-month period of assessment, demonstrating that the placebo formulations are stable in storage.

### VII. References

1. Thyagarajan A, Jones S M, Kemper A R, Pons L, Kulis M, Woo C, Yoo S, Burks A W, Shreffler W G: Basophil Suppression in Peanut Allergic Subjects undergoing Peanut Oral Immunotherapy (OIT), Journal of Allergy and Clinical Immunology 2009, 123:S214-S214.
2. Burks A W: Early peanut consumption: postpone or promote?, J Allergy Clin Immunol 2009, 123:424-425.
3. Burks W: Diagnosis of allergic reactions to food, Pediatr Ann 2000, 29:744-752.
4. Burks W: Current understanding of food allergy, Ann N Y Acad Sci 2002, 964:1-12.
5. Burks W: Skin manifestations of food allergy, Pediatrics 2003, 111:1617-1624.
6. Burks W: Peanut allergy: a growing phenomenon, J Clin Invest 2003, 111:950-952.
7. Burks W: Food allergens, Clin Allergy Immunol 2004, 18:319-337.
8. Maloney J M, Sampson H A, Sicherer S H, Burks W A: Food allergy and the introduction of solid foods to infants: a consensus document, Ann Allergy Asthma Immunol 2006, 97:559-560; author reply 561-552.
9. Skripak J M, Wood R A: Mammalian milk allergy: avoidance strategies and oral desensitization, Curr Opin Allergy Clin Immunol 2009, 9:259-264.
10. Buchanan A D, Green T D, Jones S M, Scurlock A M, Christie L, Althage K A, Steele P H, Pons L, Helm R M, Lee L A, Burks A W: Egg oral immunotherapy in nonanaphylactic children with egg allergy, J Allergy Clin Immunol 2007, 119:199-205.
11. Sicherer S H, Wood R A, Stablein D, Burks A W, Liu A H, Jones S M, Fleischer D M, Leung D Y, Grishin A, Mayer L, Shreffler W, Lindblad R, Sampson H A: Immunologic features of infants with milk or egg allergy enrolled in an observational study (Consortium of Food Allergy Research) of food allergy). J Allergy Clin Immunol 2010, 125:1077-1083 e1078. PMCID: PMC2868273
12. Hofmann A M, Scurlock A M, Jones S M, Palmer K P, Lokhnygina Y, Steele P H, Kamilaris J, Burks A W: Safety of a peanut oral immunotherapy protocol in children with peanut allergy, J Allergy Clin Immunol 2009, 124:286-291, 291 e281-286. PMCID: PMC2731305.
13. Joshi, P., S. Mofidi, and S. H. Sicherer, Interpretation of commercial food ingredient labels by parents of food-allergic children. J Allergy Clin Immunol, 2002, 109(6): p. 1019-21.
14. Altschul, A. S., et al., Manufacturing and labeling issues for commercial products: relevance to food allergy. J Allergy Clin Immunol, 2001, 108(3): p. 468.
15. Vierk, K., et al., Recalls of foods containing undeclared allergens reported to the US Food and Drug Administration, fiscal year 1999. J Allergy Clin Immunol, 2002, 109(6): p. 1022-6.
16. Sicherer, S. H., A. W. Burks, and H. A. Sampson, Clinical features of acute allergic reactions to peanut and tree nuts in children. Pediatrics, 1998. 102(1): p. e6
17. Sampson, H. A., et al., Symposium on the definition and management of anaphylaxis: summary report. J Allergy Clin Immunol, 2005. 115(3): p. 584-91.
18. Skolnick, H. S., et al., The natural history of peanut allergy. J Allergy Clin Immunol, 2001. 107(2): p. 367-74.
19. Bock, S. A., A. Munoz-Furlong, and H. A. Sampson, Fatalities due to anaphylactic reactions to foods. J Allergy Clin Immunol, 2001. 107(1): p. 191-3.
20. Frew, A. J., 25. Immunotherapy of allergic disease. J. Allergy Clin Immunol, 2003. 111(2 Suppl): p. S712-9.
21. Wilson, D. R., M. T. Lima, and S. R. Durham, Sublingual immunotherapy for allergic rhinitis: systematic review and meta-analysis. Allergy, 2005. 60(1): p. 4-12.
22. Lehrer, S. B., et al., Immunotherapy for food allergies. Past, present, future. Clin Rev Allergy Immunol, 1999. 17(3): p. 361-81.
23. Oppenheimer, J. J., et al., Treatment of peanut allergy with rush immunotherapy. J Allergy Clin Immunol, 1992. 90(2): p. 256-62.
24. Nelson, H. S., et al., Treatment of anaphylactic sensitivity to peanuts by immunotherapy with injections of aqueous peanut extract. J Allergy Clin Immunol, 1997. 99(6 Pt 1): p. 744-51.
25. Kim, E. H., et al., Sublingual immunotherapy for peanut allergy: clinical and immunologic evidence of desensitization. J Allergy Clin Immunol, 2011. 127(3): p. 640-6 e1.
26. Varshney, P., et al., A randomized controlled study of peanut oral immunotherapy: clinical desensitization and modulation of the allergic response. J Allergy Clin Immunol, 2011. 127(3): p. 654-60.
27. Bousquet, J., Primary and secondary prevention of allergy and asthma by allergen therapeutic vaccines. Clin Allergy Immunol, 2004. 18: p. 105-14.
28. Kapsenberg, M. L., et al., The paradigm of type 1 and type 2 antigen-presenting cells. Implications for atopic allergy. Clin Exp Allergy, 1999. 29 Suppl 2: p. 33-6
29. Secrist, H., R. H. DeKruyff, and D. T. Umetsu, Interleukin 4 production by CD4+ T cells from allergic individuals is modulated by antigen concentration and antigen-presenting cell type. J Exp Med, 1995. 181(3): p. 1081-9.
30. Blumchen, K., et al., Oral peanut immunotherapy in children with peanut anaphylaxis. J Allergy Clin Immunol, 2010. 126(1): p. 83-91 e1.
31. Jones, S. M., et al., Clinical efficacy and immune regulation with peanut oral immunotherapy. J Allergy Clin Immunol, 2009. 124(2): p. 292-300, 300 e1-97.
32. Narisety, S. D., et al., Open-label maintenance after milk oral immunotherapy for IgE-mediated cow's milk allergy. J Allergy Clin Immunol, 2009. 124(3): p. 610-2.
33. Skripak, J. M., et al., A randomized, double-blind, placebo-controlled study of milk oral immunotherapy for cow's milk allergy. J Allergy Clin Immunol, 2008. 122(6): p. 1154-60.
34. Bock, S. A. and F. M. Atkins, Patterns of food hypersensitivity during sixteen years of double-blind, placebo-controlled food challenges. J Pediatr, 1990. 117(4): p. 561-7.
35. Burks A W, Jones S M, Wood R A, Fleischer D M, Sicherer S H, Lindblad R, Stablein D, Henning A K, Vickery B P, Liu A H, Scurlock A M, Shreffler W G, Plaut M, and Sampson H A for the Consortium of Food Allergy Research. Oral Immunotherapy for Treatment of Egg Allergy in Children. N Engl J Med, 2012 Jul. 19; 367:233-243.
36. Bock S A, Sampson H A, Atkins F M, Zeiger R S, Lehrer S, Sachs M et al. Double-blind, placebo-controlled food challenge (DBPCFC) as an office procedure: A manual. J Allergy Clin Immunol 1988; 82:986-97.
37. Sicherer S H. Food allergy: when and how to perform oral food challenges, Pediatr Allergy Immunol 1999; 10(4):226-34.

### VIII. Abbreviations

| | |
|---|---|
| ACE | Angiotensin-converting enzyme inhibitors |
| AE | Adverse Event |
| Ag | Antigen |
| ARB | Angiotensin-receptor blockers |
| ARC | Allergen Research Corporation |
| CFR | US Code of Federal Regulations |
| CFSE | Carboxyfluorescein Succinimidyl Ester |
| CPNA | Characterized Peanut Allergen |
| CRC | Clinical Research Center |
| CRF | Case Report Form |
| CTC | Common Toxicity Criteria |
| DBPCFCs = OFC | Double-Blind, Placebo-Controlled Food Challenges - |
| | Oral Food Challenge |
| DSMB | Data Safety Monitoring Board |
| EC | Ethics Committee |
| FDA | US Food and Drug Administration |
| cGCP | Current Good Clinical Practice |
| ICH | International Conference on Harmonization |
| IFNg | Interferon Gamma |
| IgA | Immunoglobulin A |
| IgE | Immunoglobulin E |
| IgG | Immunoglobulin G |
| IL | Interleukin |
| IND | Investigational New Drug Application |
| IRB | Institutional Review Board |
| kU_{A}/L | Kilounits of Antibody per Liter |
| MedDRA | Medical Dictionary for Regulatory Activities |
| OFC = DBPCFC | Oral Food Challenge - Double Blind Placebo |
| | Controlled Food Challenge |
| OIT | Oral Immunotherapy |
| PI | Principal Investigator |
| PST | Prick Skin Tests |
| SAE | Serious Adverse Event |
| SAR | Serious Adverse Reaction |
| SUSAR | Suspected Serious Adverse Reaction |
| Th1 | T Helper 1 |
| Th2 | T Helper 2 |
| TLR | Toll-like Receptor |
| Tr1 | T Regulatory 1 |
| Tregs | Regulatory T cells |

### IX. Conclusion

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All publications, patents, and patent applications mentioned in this specification are herein incorporated by reference to the same extent as if each individual publication, patent, or patent application was specifically and individually indicated to be incorporated by reference.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while process steps, formulation components or functions are presented in a given order, alternative embodiments may include these in a different order, or substantially concurrently. The teachings of the disclosure provided herein can be applied to other compositions, not only the compositions described herein. The various embodiments described herein can be combined to provide further embodiments.

Specific elements of any of the foregoing embodiments can be combined or substituted for elements in other embodiments. Furthermore, while aspects associated with certain embodiments of the disclosure have been described in the context of these embodiments, other embodiments may also exhibit such aspects, and not all embodiments need necessarily exhibit such aspects to fall within the scope of the disclosure. Accordingly, the disclosure is not limited, except as by the appended claims.

1. A placebo composition that is matched in color and texture compared to a composition comprising peanut flour, the placebo composition comprising one or more glidants, one or more lubricants, one or more diluents, and optionally, one or more colorants.

2. (canceled)

3. The placebo composition of claim 1, wherein the one or more colorants is a caramel color.

4. The placebo composition of claim 3, wherein the caramel color comprises Sensient (05439) or Sethness (0C234).

5. The placebo composition of claim 3, wherein the caramel color comprises ammonia caramel, baker's caramel, confectioner's caramel, or beer caramel.

6. (canceled)

7. The placebo composition of claim 1, wherein the placebo composition is for use in clinical trials as a placebo for treatment of peanut allergy.

8. (canceled)

9. The placebo composition of claim 1, wherein the placebo composition is formulated for oral administration.

10. The placebo composition of claim 1, wherein the placebo composition is formulated as a capsule, a tablet, a mini-tablet, a powder, or a sprinkle.

11. The placebo composition of claim 1, wherein the placebo composition is a capsule comprising a hydroxypropyl methyl cellulose (HPMC) capsule shell.

12. The placebo composition of claim 1, wherein the one or more diluents is selected from the group consisting of alginic acid and salts thereof, cellulose derivatives, carboxymethylcellulose, methylcellulose, hydroxypropylmethylcellulose, hydroxyethylcellulose, hydroxypropylcellulose, ethylcellulose, microcrystalline cellulose, silicified microcrystalline cellulse, microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, sucrose, glucose, dextrose, molasses, mannitol, sorbitol, xylitol, lactose, dicalcium phosphate, a natural or synthetic gum, acacia, tragacanth, ghatti gum, mucilage of isapol husks, polyvinylpyrrolidone, larch arabogalactan, Veegum®, polyethylene glycol, waxes, sodium alginate, a starch, corn starch, potato starch, a pregelatinized starch, Colorcon (Starch 1500), National 1551, Amijel®, sodium starch glycolate, Promogel®, Explotab®, a cross-linked starch, sodium starch glycolate, a cross-linked polymer, crospovidone, a cross-linked polyvinylpyrrolidone, alginic acid or a salt of alginic acid, a clay, Veegum® HV (magnesium aluminum silicate), a gum, agar, guar, locust bean, Karaya, pectin, tragacanth, sodium starch glycolate, bentonite, a natural sponge, a surfactant, a resin, a cation-exchange resin, citrus pulp, sodium lauryl sulfate, and combinations thereof.

13. (canceled)

14. (canceled)

15. The placebo composition of claim 1, wherein the one or more glidants is selected from the group consisting of colloidal silicon dioxide (Cab-O-Sil), talc, and combinations thereof.

16. (canceled)

17. The placebo composition of claim 1, wherein the glidant comprises from about 0.01% to about 10% w/w of the composition.

18. (canceled)

19. (canceled)

20. (canceled)

21. (canceled)

22. (canceled)

23. The placebo composition of claim 1, wherein the one or more lubricants is selected from the group consisting of stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol, Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and combinations thereof.

24. (canceled)

25. (canceled)

26. The placebo composition of claim 1, wherein the placebo composition comprises one or more diluents in an amount of from about 1% to about 99% w/w, from about 60% to about 90%, or from about 5% to about 20% w/w of the placebo composition.

27. (canceled)

28. (canceled)

29. (canceled)

30. (canceled)

31. The placebo composition of claim 1, wherein the lubricant comprises from about 0.01% to about 10% w/w of the placebo composition

32. (canceled)

33. (canceled)

34. (canceled)

35. (canceled)

36. (canceled)

37. The placebo composition of claim 1, further comprising a food product.

38. The placebo composition of claim 1, wherein the one or more diluents comprises a particle size of from about 10 to about 150 μm.

39. (canceled)

40. (canceled)

41. (canceled)

42. (canceled)

43. (canceled)

44. (canceled)

45. The placebo composition of claim 1, comprising about 10% starch, about 89% microcellulose, about 0.5% Cab-o-Sil and about 0.5% magnesium stearate w/w of the composition.

46. A placebo composition comprising:
one or more glidants in an amount of from about 0.01% to about 10% w/w of the placebo composition,
one or more lubricants in an amount of from about 0.01% to about 10% w/w of the placebo composition,
one or more diluents in an amount of from about 1% to about 99% w/w of the placebo composition, and
one or more caramel colorants in an amount of from about 0.01% to about 10% w/w of the placebo composition,
wherein the placebo composition is matched in color and texture compared to a composition comprising peanut flour.

47. (canceled)

48. (canceled)

49. (canceled)

50. The placebo composition of claim 46, wherein the placebo composition is formulated for oral administration and is formulated as a capsule, a tablet, a mini-tablet, a powder, or a sprinkle.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A placebo composition formulated for oral administration and that is matched in color and texture compared to a composition comprising peanut flour, the placebo composition comprising:
one or more diluents in an amount of from about 1% to about 99% w/w of the placebo composition,
one or more caramel colorants in an amount of from about 0.01% to about 10% w/w of the placebo composition, and
a HPMC capsule shell.

68. (canceled)

69. (canceled)

70. (canceled)

71. (canceled)
